(19) United States
(12) Patent Application Publication
(10) Pub. No.: US 2022/0378716 A1
(43) Pub. Date: Dec. 1, 2022

(54) FORMULATION TO REDUCE OR PREVENT OXIDATIVE STRESS DAMAGE

(71) Applicant: CORA THERAPEUTICS INC., Toronto (CA)

(72) Inventors: Kieran MURPHY, Toronto (CA); Fergal KERINS, Toronto (CA); Mark Andrew TARNOPOLSKY, Dundas (CA)

(21) Appl. No.: 17/772,478

(22) PCT Filed: Oct. 27, 2020
(86) PCT No.: PCT/CA2020/051439

§ 371 Date: Apr. 27, 2022

Related U.S. Application Data
(60) Provisional application No. 62/926,953, filed on Oct. 28, 2019.

(51) Int. Cl. A61K 31/05; A61K 31/07; A61K 31/355; A61K 31/375; A61K 31/385; A61K 33/04

(52) U.S. Cl. CPC A61K 31/05; A61K 31/07; A61K 31/355; A61K 31/375; A61K 31/385; A61K 33/04

(57) ABSTRACT

A combination that includes: a flavonoid, for example in an amount from about 0.1 g to about 1.5 g; and one or more of: ascorbic acid, ascorbate, or a combination thereof, for example in an amount from about 0.2 g to about 2.0 g; N-acetyl cysteine, for example in an amount from about 0.10 g to about 1.2 g; alpha-lipoic acid, for example in an amount from about 0.05 g to about 0.60 g; and at least one carotenoid, for example in an amount from about 1 mg to about 50 mg. The combination may be used to mitigate or prevent nuclear DNA damage, mitochondrial DNA damage, lipid peroxidation, protein carbonylation, or any combination thereof in a subject caused by oxidative stress.

### CROSS REFERENCE TO RELATED APPLICATIONS

This application claims the benefit of priority to U.S. Provisional Application No. 62/926,953, filed on Oct. 28, 2019, which is incorporated by reference herein in its entirety.

### FIELD

The present disclosure relates to combinations of compounds that may be used to mitigate or prevent nuclear DNA damage, mitochondrial DNA damage, lipid peroxidation, protein carbonylation, or any combination thereof in a subject caused by oxidative stress.

### BACKGROUND

The following paragraphs are not an admission that anything discussed in them is prior art or part of the knowledge of persons skilled in the art.

Oxidative stress refers to a biological system's inability to detoxify reactive oxygen species, or to repair damage caused by those reactive species, at a sufficiently rapid rate compared to the rate that the reactive oxygen species or the damage is being produced. Elevated levels of reactive oxygen species, such as peroxides and free radicals, can damage proteins, lipids, and DNA.

### Introduction

The following introduction is intended to introduce the reader to this specification but not to define any invention. One or more inventions may reside in a combination or sub-combination of the apparatus elements or method steps described below or in other parts of this document. The inventors do not waive or disclaim their rights to any invention or inventions disclosed in this specification merely by not describing such other invention or inventions in the claims.

Oxidative stress may result from acute exposure to ionizing radiation, such as at doses larger than about 0.05 mSv over a period of time shorter than 5 hours. The ionizing radiation may be from a surgical procedure where X-ray guidance, computed tomography (CT), a radioactive tracer, or another source of ionizing radiation is used; from a medical imaging procedure such as X-ray, CT, or dental X-ray; from a medical procedure where a radioactive material, a contrast agent, or a radio-isotope is used; or from an environmental exposure, such as air-travel, industrial X-ray use, or proximity to a radiation source (for example a nuclear plant, a nuclear-waste storage site, or an accidental or deliberate release of radioactive material).

Oxidative stress may result from smoking or vaping, for example, tobacco- or cannabis-products. Without wishing to be bound by theory, smoking or vaping are believed to increase oxidative stress through direct damage by reactive oxygen species and/or other radical species, the inflammatory response caused by the smoking or vaping, or both.

Elevated levels of reactive oxygen species, such as those produced under oxidative stress, can damage cells. Cumulative cell damage is correlated with increased risk of various diseases and conditions, such as cancers, cataracts, and vascular and cardiovascular conditions. Reducing the average level of oxidative stress in an individual, by even a small amount, may be beneficial since prolonged, low-grade inflammation may overwhelm damage repair pathways and the signals that initiate such repair processes.

Since prolonged and/or cumulative exposure to ionizing radiation, smoking, or vaping may result in oxidative stress related damage to proteins, lipids, and/or DNA, it is desirable to provide a combination of compounds that reduces oxidative stress when administered to a subject.

The present disclosure provides a combination that includes: a flavonoid and one or more of: ascorbic acid or ascorbate; N-acetyl cysteine; alpha-lipoic acid; at least one carotenoid; and folic acid, folate or methylfolate. In particular examples, the present disclosure provides a combination that includes the flavonoid; the ascorbic acid or ascorbate; the N-acetyl cysteine; the alpha-lipoic acid; optionally the at least one carotenoid; and optionally the folic acid, folate or methylfolate. Some combinations according to the present disclosure may be used to mitigate or prevent nuclear DNA damage, mitochondrial DNA damage, lipid peroxidation, protein carbonylation, or any combination thereof in a subject caused by oxidative stress. Some combinations according to the present disclosure may be used to aid repair of nuclear DNA damage, mitochondrial DNA damage, lipid peroxidation, protein carbonylation, or any combination thereof in a subject.

### DETAILED DESCRIPTION

Generally, the present disclosure provides a combination that includes a flavonoid and one or more of: ascorbic acid or ascorbate; N-acetyl cysteine; alpha-lipoic acid; at least one carotenoid; and folic acid, folate or methylfolate.

It should be understood that, in the context of the present disclosure, “a combination” is not limited to formulations where all of the components of the combination are physically together. Rather, “a combination” according to the present disclosure refers to components that are physically separate but are intended to be administered to a subject within about a one hour window.

For example, a combination according to the present disclosure may be formulated so that all of the compounds of the combination are administerable to a subject together. One specific example of such a formulation is a water-dispersable powder.

In another example, a combination according to the present disclosure may physically separate water-soluble components from water-insoluble and water-unstable components. The water-soluble components may be formulated in a water-based drink, while the water-insoluble and water-unstable components may be formulated in a water-free edible gel or water-free potable liquid.

The flavonoid used in a combination according to the present disclosure may have antioxidant properties. In some examples, the flavonoid is a flavanol, such as quercetin or a quercetin derivative. In other examples, the flavonoid is a flavanonol, such as dihydroquercetin or a dihydroquercetin derivative. A combination according to the present disclosure may include a mixture of flavonoids.

A flavonoid “derivative”, such as a quercetin derivative or a dihydroquercetin derivative, may be a glycosylated flavonoid or a pro-drug flavonoid analog. Examples of glycosylated quercetin include: quercetin-3-O-paltoside, quercetin-3-O-glucoside, quercetin-3-O-rutinoside, quercetin-3-O-galactoside, and quercetin-3-O-rhamnoside. Examples of glycosylated dihydroquercetin include: dihydroquercetin-3-O-rhamnoside; dihydroquercetin-3-O-glucoside; (−)-2,3-trans-dihydroquercetin-3′-O-β-D-glucopyranoside; (2S,3S)-(−)-dihydroquercetin-3-O-β-D-glucopyranoside; (2R,3R)-dihydroquercetin-3′-O-β-D-pyranoglucoside; dihydroquercetin-4′-O-β-glucopyranoiside; (2R,3R)-dihydroquercetin-3-O-arabinoside; and (2S,3S)-dihydroquercetin-3-O-arabinoside. An example of a pro-drug flavonoid analog is an ester-modified quercetin or glycosylated quercetin. The ester-modification may be removed by an esterase or via hydrolysis to reveal the quercetin or glycosylated quercetin.

Quercetin and/or quercetin derivatives may be derived or isolated from apple skin, onions, or from the seed or flower of Sophora japonica. Exemplary methods are discussed in U.S. Pat. No. 9,101,649, which is hereby incorporated by reference. In one example, quercetin and/or quercetin derivatives may be extracted from apple skin, such as from a dried apple skin powder, using a food-grade solvent, for example ethanol. The apple skins may be sonicated during the extraction. Extracted quercetin and/or quercetin derivatives may be separated from the remaining solids, and optionally concentrated, dried, and/or frozen. The extracted quercetin and/or quercetin derivatives may be purified, such as by column chromatography.

In a combination according to the present disclosure: the flavonoid and the ascorbic acid or ascorbate may be in a mass/mass ratio from about 1:10 to about 5:4; the flavonoid and the N-acetyl cysteine may be in a mass/mass ratio from about 1:5 to about 5:1; the flavonoid and the alpha-lipoic acid may be in a mass/mass ratio from about 1:5 to about 5:1; the flavonoid and the at least one carotenoid may be in a mass/mass ratio from about 50:1 to about 10:1; the flavonoid and the folic acid, folate or methylfolate may be in a mass/mass ratio from about 10,000:1 to about 100:1; or any combination thereof.

In a combination according to the present disclosure: the flavonoid may be in an amount from about 0.1 g to about 1.5 g; the ascorbic acid or ascorbate may be in an amount from about 0.2 g to about 2.0 g; the N-acetyl cysteine may be in an amount from about 0.10 g to about 1.2 g; the alpha-lipoic acid may be in an amount from about 0.05 g to about 0.60 g; the at least one carotenoid may be in an amount from about 10 mg to about 50 mg; the folic acid, folate or methylfolate may be in an amount from about 100 μg to about 400 μg; or any combination thereof.

Particular examples of a combination according to the present disclosure include: the flavonoid; the ascorbic acid or ascorbate; the N-acetyl cysteine; and the alpha-lipoic acid.

A combination according to the present disclosure may include at least one carotenoid, which may be a carotene compound, such as beta-carotene; a xanthophyll compound, such as lutein or zeaxanthin; or a combination thereof. A combination that includes at least one carotenoid may mitigate or prevent nuclear DNA damage, mitochondrial DNA damage, lipid peroxidation, protein carbonylation, or any combination thereof in epithelial cells, such as vascular epithelial cells, ocular epithelial cells, or both; connective tissue cells; muscle tissue cells; nerve cells; or any combination thereof. The DNA damage may include double strand breaks, single strand breaks, oxidative lesions such as 8-OH-2-deoxyguanosine, or combinations thereof.

In the context of the present disclosure, mitigating or preventing nuclear DNA damage, mitochondrial DNA damage, lipid peroxidation, protein carbonylation, or any combination thereof should be understood to mean that, statistically, a population of subjects that is administered a combination according to the present disclosure has less nuclear DNA damage, mitochondrial DNA damage, lipid peroxidation, protein carbonylation, or a combination thereof, in at least one tissue- or cell-type, after an oxidative stress event when compared to an otherwise statistically identical population of subjects that is not administered the combination. For example, mitigating or preventing lipid peroxidation should be understood to mean that, statistically, a population of subjects that is administered a combination according to the present disclosure has less lipid peroxidation in skin cells after an oxidative stress event when compared to the primary fibroblasts of an otherwise statistically identical population of subjects that is not administered the combination.

In the context of the present disclosure, nuclear DNA damage is evaluated by counting the total number of double strand DNA breaks per cell in peripheral blood mononuclear cells (PBMCs) using three-dimensional microscopy and fluorescently labeled gamma-H2AX protein. Gamma-H2AX protein is a phospho-isoform of histone H2A, which is phosphorylated during detection of double strand DNA breaks to facilitate recruitment of repair proteins. To quantify the nuclear DNA damage, the gamma-H2AX is fluorescently tagged and used to count the total number of double strand breaks per cell via fluorescence microscopy. The gamma-H2AX is fluorescently tagged using an Abcam Gamma H2A.X staining kit (ab242296) according to the manufacturer's protocols.

In the context of the present disclosure, mitochondrial DNA damage is evaluated using long range PCR (LR-PCR) and digital PCR. The LR-PCR method uses extracted mitochondrial DNA (mtDNA) that is amplified using high fidelity DNA polymerase mediated long range polymerase chain reaction (PCR) methodology as discussed by Gianni P., et al. in Exp. Gerontol. 2004 September; 39(9): 1391-400. The mitochondrial DNA damage is quantified as the amount of full length amplifiable mtDNA AND the abundance of mtDNA deletions.

In the context of the present disclosure, total DNA damage is measured using 8-hydroxy-2′-deoxyguanosine (8-OHdG) in cellular lysates using an 8-Oxo detection Elisa kit (Cayman chemical 589320) according to the manufacturer's protocol.

Lipid peroxidation refers to the oxidative degradation of lipids, which form reactive aldehydes such as malondialdehyde (MDA) and 4-hydroxynonenal (4-HNE). MDA and 4-HNE are commonly used as markers of lipid peroxidation. In the context of the present disclosure, lipid peroxidation is evaluated by a fluormetric Western-blot assay to quantify the amounts of malondialdehyde (MDA) or 4-hydroxynonenal (4-HNE) in the sample. The lipid peroxidation is measured in cell lysates using a Western blotting approach using an anti-4-HNE antibody, as discussed by Kitaoka, Y., et al. in Mol Genet Metab. 2013 November; 110(3):297-302. It should be understood that mitigating or preventing lipid peroxidation would be achieved as long as the amount of MDA, the amount of 4-HNE, or the combined amount of MDA and 4-HNE was statistically lower in the population of subjects that is administered a combination according to the present disclosure, as compared to the statistically identical population of subjects that is not administered the combination.

Protein carbonylation is a form of protein oxidation in which reactive ketones and/or aldehydes are formed. In the context of the present disclosure, protein carbonylation is evaluated by a fluorometric assay to quantify the amounts of ketones and/or aldehydes in the sample. The ketones and/or aldehydes are first reacted with 2,4-dinitrophenylhydrazine (DNPH) to form hydrazones, which are then quantified using Western blotting, as discussed by Kitaoka, Y., et al. in Mol Genet Metab. 2013 November ; 110(3):297-302.

The oxidative stress event may be, for example, an acute exposure to ionizing radiation, or repeated exposures to sub-acute levels of ionizing radiation. The tissue- or cell-type may be bone marrow, skeletal muscle (for example: quadriceps), skin tissue, lung tissue, or heart tissue.

A combination according to the present disclosure may include folic acid, folate, or methylfolate. A combination that includes folic acid, folate, or methylfolate may be used to mitigate or prevent nuclear DNA damage caused by oxidative stress from a stroke and/or heart attack in a subject.

A combination according to the present disclosure may include iodide, for example potassium iodide. The flavonoid and the iodide may be in a mass/mass ratio from about 10:1 to about 1:1. The iodide may be in an amount for about 30 mg to about 160 mg. A combination that includes iodide may be used to reduce or prevent absorption of radioactive iodine by a subject, such as by the subject's thyroid gland.

A combination according to the present disclosure may include an anti-oxidizing glycosylated or un-glycosylated isoflavone, such as genistein or daidzein. A combination that includes an anti-oxidizing glycosylated or un-glycosylated isoflavone may mitigate or prevent nuclear DNA damage caused by oxidative stress from skin inflammation induced by exposure to UV radiation, such as solar UV radiation.

A combination according to the present disclosure may include an anti-oxidizing phytocannabinoid, such as cannabidiol (CBD). A combination that includes an anti-oxidizing phytocannabinoid may mitigate or prevent nuclear DNA damage caused by oxidative stress from skin inflammation induced by exposure to UV radiation, such as solar UV radiation. A combination that includes CBD may be formulated for transdermal and oral administration, with the CBD and the flavonoid formulated for transdermal administration and the remaining components of the combination formulated for oral administration. The transdermal formulation, containing the CBD, may provide an analgesic effect.

A combination according to the present disclosure may include a radio-imaging contrast agent, such as barium sulfate or a gadolinium- or iodine-based contrast agent. Radio-imaging contrast agents may effect DNA breaks in a patient, for example when the patient is being treated with chemotherapy and is subjected to X-rays. A combination that includes a radio-imaging contrast agent may mitigate or prevent nuclear DNA damage caused by a medical imaging procedure. For example, a combination of barium sulfate, a flavonoid such as quercetin, ascorbic acid or ascorbate, N-acetyl cysteine, and alpha-lipoic acid may be formulated for oral administration. A patient may be administered such a formulation in advance of a gastrointestinal (GI) fluoroscopy, or in advance of a CT scan. In another example, a gadolinium-based contrast agent may be formulated for intravenous administration and a mixture of a flavonoid such as quercetin, ascorbic acid or ascorbate, N-acetyl cysteine, and alpha-lipoic acid may be formulated for oral administration.

A combination according to the present disclosure may be formulated for oral administration, or transdermal administration, or a mixture of both oral and transdermal administration.

Some flavonoids that be included in combinations according to the present disclosure are unstable when exposed to water for prolonged periods of time, such for days, weeks or months. Formulations that include such water-unstable flavonoids are substantially water-free, though the substantially water-free formulations may be mixed with water before administration, such as up to an hour before administration. In the context of the present disclosure, “substantially water-free” should be understood to mean that no more than 10 mol % of the flavonoid in the formulation degrades after one week due to water present in the formulation. Some formulations may include measurable amounts of water while still being considered substantially water-free. For example, a carbohydrate-based gel may be considered substantially water-free if it formed enough hydrogen bonds with the water present in the formulation to prevent the water from degrading more than 10 mo l% of the flavonoid, as measured after one week.

A formulation for oral administration may be a water-dispersible powder, a tablet, a capsule, an edible gel, a potable liquid, or any combination thereof. A water-dispersible powder may include, for example, microcrystalline cellulose (MCC), calcium chloride, or both. Microcrystalline cellulose may aid in the dispersion of water-insoluble components, such as a water-insoluble flavonoid, in a drinkable liquid. A tablet may include MCC, calcium chloride, magnesium stearate, sodium carbonate, bicarbonate and/or citric acid.

In one example, a formulation for oral administration may be a combination of: (i) an aqueous-based edible gel or potable liquid comprising one or more water-soluble components, such as the ascorbic acid or ascorbate, the N-acetyl cysteine, and/or the folic acid, folate, or methylfolate, and (ii) a substantially water-free powder, water-free edible gel or water-free potable liquid comprising at least the flavonoid, and optionally the alpha-lipoic acid and/or the carotenoid.

In a particular example, a drinkable liquid that includes ascorbic acid or ascorbate, N-acetyl cysteine, and folic acid, folate or methylfolate may be provided with a packet containing a substantially water-free powder or gel that includes quercetin, alpha-lipoic acid, and beta-carotene. The powder or gel may be mixed with the drinkable liquid and the resulting mixture may be ingested.

A combination according to the present disclosure may be formulated in a dose that is suitable for a particular risk profile associated with a particular exposure to ionizing radiation. For example, the combination of compounds may be formulated in a dose suitable for a patient who will be exposed to a particular dose of X-rays.

Alternatively, the combination of compounds may be formulated in a dose that can be combined with additional doses depending on the risk profile associated with an exposure to ionizing radiation. For example, the combination of compounds may be formulated in a dose where administration of a single dose is suitable for a person with a low-risk profile, and administration of multiple doses, such as two or three doses, is suitable for a person with a high-risk profile.

In the context of the present disclosure, a person with a “low-risk” profile is someone who might be exposed to from about 0.05 mSv to about 5 mSv over a 5-hour period of time. A person with a low-risk profile may be, for example, a hospital employee, such as a doctor or a nurse, who is exposed on a daily or near-daily basis to low-levels of ionizing radiation when they are at work. Such a person with a low-risk profile may be administered a prophylactic dose of the combination on days that they go to work. In another example, a person with a low-risk profile may be a traveler, pilot or flight attendant flying for at least 6 hours. Such a person with low-risk profile may be administered a dose of the combination starting at from 0 to about 4 hours before the start of the flight, and a subsequent dose every about 6 to about 12 hours.

In the context of the present disclosure, a person with a “high-risk” profile is someone who might be exposed to over 5 mSv over a 5-hour period of time. A person with a high-risk profile may be, for example, a person exposed to a high-level of ionizing radiation, such as due to accidental exposure of radioactive material.

In some examples, a combination according to the present disclosure includes about 2.0 g of ascorbic acid or ascorbate, about 1.2 g of N-acetyl cysteine, about 0.6 g of alpha-lipoic acid, about 0.5 g of quercetin, about 30 mg of beta-carotene, and about 400 μg of folic acid, folate, or methylfolate. Such a combination may be formulated for oral administration to a subject, for example in a water-dispersible powder that may be mixed with a water-based drink, for example a barium sulfate suspension, to provide a drinkable formulation. The formulation may be ingested by the subject about 2 hours in advance of a planned exposure to ionizing radiation, such as a medical imaging procedure.

In some examples, a combination according to the present disclosure includes about 0.8 g of ascorbic acid or ascorbate, about 0.6 g of N-acetyl cysteine, about 0.5 to about 0.6 g of alpha-lipoic acid, about 0.5 g of quercetin, about 15 mg of beta-carotene, and about 200 μg to about 400 μg of folic acid, folate, or methylfolate. Such a combination may be formulated for oral or transdermal administration to a subject with a “low-risk” profile. The combination may be, for example, administered to the subject about an hour or two before they go to work, or fly.

In some examples, a combination according to the present disclosure includes about 0.8 g of ascorbic acid or ascorbate, about 0.6 g of N-acetyl cysteine, about 0.6 g of alpha-lipoic acid, about 0.5 g of quercetin, about 15 mg of beta-carotene, about 400 μg of folic acid, folate, or methylfolate, and about 130 mg of potassium iodide. Such a combination may be formulated for oral or transdermal administration to a subject who may be exposed to depleted or enriched uranium. The subject may be, for example, an individual participating in clean-up of a radioactive industrial accident; or an individual exposed to armaments containing or releasing depleted or enriched uranium.

In some examples, a combination according to the present disclosure includes about 1.0 g of ascorbic acid or ascorbate, about 0.6 g of N-acetyl cysteine, about 0.3 g of alpha-lipoic acid, about 0.25 g of quercetin, about 15 mg of beta-carotene, and about 200 μg of folic acid, folate, or methylfolate. Such a combination may be formulated for oral administration, such as in a water-dispersible powder. One dose of the combination may be suitable for daily or near-daily administration to a person with a low-risk profile, while two or three doses of the combination may be suitable for a one-time administration, or for daily administration during a short period of time (such as from about 1 week to about 6 months), to a person with a high-risk profile.

In some examples, a combination according to the present disclosure includes about 2.0 g of ascorbic acid or ascorbate, about 1.2 g of N-acetyl cysteine, about 0.6 g of alpha-lipoic acid, about 0.5 g of quercetin, about 30 mg of beta-carotene, and 400 μg of folic acid, folate, or methylfolate. Such a combination may be formulated for oral and transdermal administration to a subject who was, or is expected to be, exposed to UV irradiation. The portion of the combination formulated for oral administration may include the ascorbic acid or ascorbate, the N-acetyl cysteine, the alpha-lipoic acid, the beta-carotene, and the folic acid, folate, or methylfolate. The portion of the combination formulated for transdermal administration, such as in a cream or ointment, may include the quercetin and optionally cannabidiol.

In the preceding description, for purposes of explanation, numerous details are set forth in order to provide a thorough understanding of the examples. However, it will be apparent to one skilled in the art that these specific details are not required. Accordingly, what has been described is merely illustrative of the application of the described examples and numerous modifications and variations are possible in light of the above teachings.

Since the above description provides examples, it will be appreciated that modifications and variations can be effected to the particular examples by those of skill in the art. Accordingly, the scope of the claims should not be limited by the particular examples set forth herein, but should be construed in a manner consistent with the specification as a whole.

What is claimed is:

1. A combination comprising:
a flavonoid, for example in an amount from about 0.1 g to about 1.5 g; and
one or more of:
ascorbic acid, ascorbate, or a combination thereof, for example in an amount from about 0.2 g to about 2.0 g;
N-acetyl cysteine, for example in an amount from about 0.10 g to about 1.2 g;
alpha-lipoic acid, for example in an amount from about 0.05 g to about 0.60 g; and
at least one carotenoid, for example in an amount from about 1 mg to about 50 mg.

2. The combination according to claim 1, comprising:
the flavonoid and the ascorbic acid, ascorbate or combination thereof in a mass/mass ratio from about 1:10 to about 5:4;
the flavonoid and the N-acetyl cysteine in a mass/mass ratio from about 1:5 to about 5:1;
the flavonoid and the alpha-lipoic acid in a mass/mass ratio from about 1:5 to about 5:1;
the flavonoid and the at least one carotenoid in a mass/mass ratio from about 200:1 to about 10:1; or
any combination thereof.

3. The combination according to claim 1 or 2, wherein the combination comprises:
the flavonoid;
the ascorbic acid or ascorbate;
optionally the N-acetyl cysteine;
the alpha-lipoic acid; and
optionally the at least one carotenoid.

4. The combination according to any one of claims 1 to 3, wherein the flavonoid is a flavonol, such as quercetin or a quercetin derivative; or a flavanonol, such as dihydroquercetin or a dihydroquercetin derivative.

5. The combination according to claim 4, wherein the quercetin derivative is a glycosylated quercetin.

6. The combination according to claim 5, wherein the glycosylated quercetin is quercetin-3-O-paltoside, quercetin-3-O-glucoside, quercetin-3-O-rutinoside, quercetin-3-O-galactoside, or quercetin-3-O-rhamnoside.

7. The combination according to any one of claims 1 to 6, wherein the at least one carotenoid comprises:
a carotene compound, such as beta-carotene;
a xanthophyll compound, such as lutein, astaxanthin, zeaxanthin, or a combination thereof; or
a combination thereof.

8. The combination according to any one of claims 1 to 7, further comprising folic acid, folate, methylfolate, or a combination thereof, for example in an amount from about 100 μg to about 400 μg.

9. The combination according to claim 8, wherein the flavonoid and the folic acid, folate, methylfolate, or combination thereof are in a mass/mass ratio from about 10,000:1 to about 100:1.

10. The combination according to any one of claims 1 to 9, further comprising iodide, for example potassium iodide in an amount for about 30 mg to about 160 mg.

11. The combination according to claim 10, wherein the flavonoid and the iodide are in a mass/mass ratio from about 10:1 to about 1:1.

12. The combination according to any one of claims 1 to 9, further comprising an anti-oxidizing glycosylated or un-glycosylated isoflavone, such as genistein or daidzein.

13. The combination according to any one of claims 1 to 9, further comprising an anti-oxidizing phytocannabinoid, such as cannabidiol.

14. The combination according to any one of claims 1 to 9, further comprising a radio-imaging contrast agent, such as barium sulfate or a gadolinium- or iodine-based contrast agent.

15. The combination according to any one of claims 1 to 14, comprising:
a) about 2.0 g of ascorbic acid, about 1.2 g of N-acetyl cysteine, about 0.6 g of alpha-lipoic acid, about 0.5 g of quercetin, about 30 mg of beta-carotene, and about 400 μg of folic acid;
b) about 0.8 g of ascorbic acid, about 0.6 g of N-acetyl cysteine, about 0.5 to about 0.6 g of alpha-lipoic acid, about 0.5 g of quercetin, about 15 mg of beta-carotene, and about 200 μg to about 400 μg of folic acid, and optionally about 130 mg of potassium iodide; or
c) about 1.0 g of ascorbic acid, about 0.6 g of N-acetyl cysteine, about 0.3 g of alpha-lipoic acid, about 0.25 g of quercetin, about 15 mg of beta-carotene, and about 200 μg of folic acid.

16. The combination according to any one of claims 1 to 15, further comprising Coenzyme Q10 (CoQ10), for example in an amount from about 100 mg to about 500 mg.

17. The combination according to claim 16, wherein the flavonoid and the CoQ10 are in a mass/mass ratio from about 5:1 to about 1:5, such as about 1:1.

18. The combination according to any one of claims 1 to 17, further comprising a tocopherol, such as Vitamin E, for example in an amount from about 100 mg to about 500 mg.

19. The combination according to claim 18, wherein the flavonoid and the tocopherol, for example Vitamin E, are in a mass/mass ratio from about 5:1 to about 1:5, such as about 1:1.

20. The combination according to any one of claims 1 to 19, further comprising selenium, selenomethionine, selenocysteine, a selenate salt, or any combination thereof for example in an amount from about 20 μg to about 500 μg.

21. The combination according to claim 20, wherein the flavonoid and the selenium, selenomethionine, selenocysteine, a selenate salt, or any combination thereof are in a mass/mass ratio from about 10,000:1 to about 100:1, such as about 2000:1.

22. The combination according to any one of claims 1 to 21, further comprising Vitamin B, such as Vitamin B12, for example in an amount from about 10 μpg to about 250 μg.

23. The combination according to claim 22, wherein the flavonoid and the Vitamin B, for example Vitamin B12, are in a mass/mass ratio from about 20,000:1 to about 100:1, such as about 4,000:1.

24. The combination according to any one of claims 1 to 14, comprising:
quercetin; Coenzyme Q10; alpha-lipoic acid; Vitamin E; and ascorbic acid, ascorbate, or both ascorbic acid and ascorbate; and
optionally folic acid, folate, methylfolate, Vitamin B12, or any combination thereof;
optionally beta-carotene, lutein, astaxanthin, zeaxanthin or any combination thereof;
optionally selenium, selenomethionine, selenocysteine, a selenate salt, or any combination thereof;
optionally iodide, such as potassium iodide.

25. The combination according to claim 24, comprising:
about 200 mg of quercetin;
about 200 mg of Coenzyme Q10;
about 200 mg of alpha-lipoic acid;
about 200 mg of Vitamin E;
about 200 mg of ascorbic acid or ascorbate, or a combination thereof;
about 4 mg of astaxanthin;
about 2.5 mg of zeaxanthin;
about 400 μg of folic acid, folate, methylfolate, or a combination thereof;
about 100 μg of selenium; and
about 50 μg of Vitamin B12.

26. The combination according to any one of claims 1 to 25, wherein the combination is formulated for oral administration, transdermal administration, or is formulated for a mixture of oral and transdermal administration.

27. The combination according to claim 26 wherein the formulation for oral administration is: a powder, a tablet, a capsule, an edible gel, a potable liquid, or any combination thereof.

28. The combination according to claim 27 wherein the formulation for oral administration is a combination of:
an aqueous-based edible gel or potable liquid comprising one or more water-soluble components, such as the ascorbic acid or ascorbate, the N-acetyl cysteine, and/or the folic acid, folate or methylfolate, and
a substantially water-free powder, substantially water-free edible gel or substantially water-free potable liquid comprising the flavonoid, and optionally the alpha-lipoic acid and/or the carotenoid.

29. The combination according to claim 27 wherein the formulation for oral administration is a water-dispersible powder, an effervescent tablet, or a potable liquid formulated to be mixed with a water-based drink.

30. The combination according to claim 26 wherein the formulation for transdermal administration is a topical cream or ointment, or a transdermal patch.

31. The combination according to any one of claims 26 to 30 wherein the combination formulated for a mixture of oral and transdermal administration comprises (i) an oral formulation that includes one or more water-soluble components, such as the ascorbic acid or ascorbate, the N-acetyl cysteine, and/or the folic acid, folate, or methylfolate, and (ii) a transdermal formulation that includes the flavonoid, and optionally the alpha-lipoic acid and/or the carotenoid.

32. The combination according to claim 24 or 25, wherein the combination is formulated for oral administration as a powder, one or more tablets, or one or more capsules.

33. The combination according to claim 32, wherein the combination is formulated for oral administration of two to five capsules; for example wherein the combination is formulated for oral administration of three capsules, each capsule comprising:
about 67 mg of quercetin;
about 67 mg of Coenzyme Q10;
about 67 mg of alpha-lipoic acid;
about 67 mg of Vitamin E;
about 67 mg of ascorbic acid or ascorbate, or a combination thereof;
about 1.3 mg of astaxanthin;
about 0.83 mg of zeaxanthin;
about 133 μg of folic acid, folate, methylfolate, or a combination thereof;
about 33 μg of selenium; and
about 17 μg of Vitamin B12.

34. The combination according to any one of claims 1 to 33, for use in mitigating or preventing: nuclear DNA damage, mitochondrial DNA damage, lipid peroxidation, protein carbonylation, or any combination thereof in a subject caused by oxidative stress.

35. The combination for use according to claim 34, wherein the nuclear DNA damage, the mitochondrial DNA damage, the lipid peroxidation, or the protein carbonylation is found in epithelial cells of the subject, such as in cardiovascular epithelial cells or corneal epithelial cells; connective tissue cells of the subject; muscle tissue cells of the subject; nerve cells of the subject; or any combination thereof.

36. The combination for use according to claim 35, wherein the epithelial cells are cardiovascular epithelial cells or corneal epithelial cells.

37. The combination for use according to any one of claims 34 to 36, wherein the oxidative stress is from exposure to ionizing radiation, such as from a medical imaging procedure, release of nuclear material, or air travel, or from skin inflammation induced by exposure to UV radiation.

38. The combination for use according to any one of claims 34 to 36, wherein the oxidative stress is from an elevated exposure to a reactive oxygen species from smoking or vaping a tobacco- or cannabis-product.

39. Use of the combination according to any one of claims 1 to 33, for mitigating or preventing nuclear DNA damage, mitochondrial DNA damage, lipid peroxidation, protein carbonylation, or any combination thereof in a subject caused by oxidative stress.

40. The use according to claim 39, wherein the nuclear DNA damage, the mitochondrial DNA damage, the lipid peroxidation, or the protein carbonylation is found in epithelial cells of the subject; connective tissue cells of the subject; muscle tissue cells of the subject; nerve cells of the subject; or any combination thereof.

41. The use according to claim 40, wherein the epithelial cells are cardiovascular epithelial cells or corneal epithelial cells.

42. The use according to any one of claims 39 to 41, wherein the oxidative stress is from exposure to ionizing radiation, such as from a medical imaging procedure, release of nuclear material, or air travel, or from skin inflammation induced by exposure to UV radiation.

43. The use according to any one of claims 39 to 41, wherein the oxidative stress is from an elevated exposure to a reactive oxygen species from smoking or vaping a tobacco- or cannabis-product.

44. A method of mitigating or preventing nuclear DNA damage, mitochondrial DNA damage, lipid peroxidation, protein carbonylation, or any combination thereof in a subject caused by oxidative stress, the method comprising administering to the subject the combination according to any one of claims 1 to 33.

45. The method according to claim 44, wherein the nuclear DNA damage, the mitochondrial DNA damage, the lipid peroxidation, or the protein carbonylation is found in epithelial cells of the subject; connective tissue cells of the subject; muscle tissue cells of the subject; nerve cells of the subject; or any combination thereof.

46. The method according to claim 45, wherein the epithelial cells are cardiovascular epithelial cells or corneal epithelial cells.

47. The method according to any one of claims 44 to 46, wherein the oxidative stress is from exposure to ionizing radiation, such as from a medical imaging procedure, release of nuclear material, or air travel, or from skin inflammation induced by exposure to UV radiation.

48. The method according to any one of claims 44 to 46, wherein the oxidative stress is from an elevated exposure to a reactive oxygen species from smoking a tobacco- or cannabis-product.

49. The method according to any one of claims 44 to 46, wherein the oxidative stress is from exposure to ionizing radiation due to air travel, and the method comprises administering to the subject the combination according to any one of claims 17 to 19 every about 6 to about 12 hours, for example starting at from 0 to about 4 hours before flight.

50. The method according to claim 49, wherein the combination comprises about 0.5 g of quercetin, about 2.0 g of ascorbic acid, about 1.2 g of N-acetyl cysteine, about 0.6 g of alpha-lipoic acid, about 30 mg of beta-carotene, and about 400 μg of folic acid.

51. The method according to claims 44 to 46, wherein the oxidative stress is from exposure to a low-level of ionizing radiation, and the method comprises administering to the subject a combination that comprises about 0.5 g of quercetin, about 0.80 g of ascorbic acid, about 0.60 g of N-acetyl cysteine, about 0.50 g of alpha-lipoic acid, about 15 mg of beta-carotene, and about 400 μg of folic acid.
